(19) United States
(12) Patent Application Publication
(10) Pub. No.: US 2022/0008219 A1
(43) Pub. Date: Jan. 13, 2022

(54) Functional Positioning for Knee Planning

(71) Applicant: Howmedica Osteonics Corp., Mahwah, NJ (US)

(72) Inventor: Scott G. Logan, Oak Ridge, NJ (US)

(21) Appl. No.: 17/373,047

(22) Filed: Jul. 12, 2021

Related U.S. Application Data
(60) Provisional application No. 63/050,924, filed on Jul. 13, 2020.

(51) Int. Cl. A61F 2/46; A61F 2/38; G16H 20/40; G16H 50/50

(52) U.S. Cl. CPC A61F 2/46; A61F 2/3859; A61F 2/389; A61F 2002/4668; G16H 50/50; A61F 2002/4633; G16H 20/40

(57) ABSTRACT

Articular features in a joint may be characterized by fitting equations to a series of control points representative of one or more articular features on a bone of the joint. The equations may be fit to the control points by use of a regression, such as a least squares regression. The fit equations may be used to create best fit curves for the articular features. Disease states may be accounted for in deriving the best fit curves from the fit equations. An articular implant may be constructed or selected to match the best fit curves.

### CROSS-REFERENCE TO RELATED APPLICATIONS

The present application claims the benefit of the filing date of U.S. Provisional Patent Application No. 63/050,924 filed Jul. 13, 2020, the disclosure of which is hereby incorporated herein by reference.

### BACKGROUND

Joint arthroplasty, such as knee arthroplasty, frequently involves resection of articular features from one or more bones in the joint and replacement of those articular features with prosthetic implants. Individual variations in each patient's joint geometry necessitate a wide range of implant shapes and sizes. Implant selection for each joint affects outcomes for the arthroplasty such as range of motion and comfort. For example, soft tissues preserved through the arthroplasty will remain sized for the pre-operative geometry of the joint, and may therefore be strained or rendered dysfunctional by improperly sized implants.

Existing methods for selecting implants include intra-operative measures. For example, gap balancing tools may be used between two already resected bones to determine relative tensions of soft tissue on either side of the joint, thereby guiding a surgeon's choice of implant. Multiple implants of various sizes may also be trialled in an open joint before a best fit is selected an implanted. Such approaches rely on indirect indicia about the pre-operative geometry of the joint, and may therefore result in post-operative geometry that differs in form or function. Such approaches also require specialized tools to be inserted into the open joint and add time to each procedure. Pre-operative imaging poses other issues, such as difficulty in accurately locating particular bone features in the joint, such as the sulcus of the medial condyle. Further, anatomical features of imaged joints are typically given equal weight in selecting implants without regard to differences in correlation between accurate reproduction of various features by the implant and patient outcomes. Typically, equal weighting in this way leaves the ligaments surrounding the joint outside of an acceptable tension range, causes the joint to deviate from natural kinematic function, or both. Moreover, articular features of bones are frequently characterized with inaccurate or simplified assumptions about the mechanics of the joint, such as the axis about which the bones rotate or the differing reference points that various features should be measured against.

Accordingly, it would be desirable to improve the accuracy of ascertained joint geometry pre-operatively and select articular implants based on prioritized criteria.

### BRIEF SUMMARY OF THE DISCLOSURE

In one aspect, a pre-operative procedure in preparation for a knee arthroplasty may include scanning a knee. Computer models of the portions of the femur and tibia near the knee may be generated from the scan data. An initial mechanical reference frame may be defined based on the computer models. The initial mechanical reference frame is a set of reference planes: sagittal, coronal and transverse, that define a Cartesian coordinate system. With the initial mechanical reference frame identified, a femoral mechanical reference frame may be established. The posterior femoral condyles may be used to orient and position the coronal plane. Then, a distal plane corresponding to the transverse plane may be established normal to the coronal plane and tangent to with the most prominent distal end of the medial condyle.

In an alternative to the above, the femoral mechanical reference frame may be defined in view of the epicondylar axis of the femur, the epicondylar axis passing through a prominence of the lateral epicondyle and the sulcus of the medial epicondyle. For example, a transverse femoral tangent plane may be defined parallel to the epicondylar axis and tangent to a distal most point on the medial femoral condyle. A coronal femoral tangent plane may be defined normal to the transverse femoral tangent plane, parallel to the epicondylar axis, and tangent to a posterior most point of either the medial femoral condyle or the lateral femoral condyle. A first sagittal femoral intersect plane may be normal to the transverse femoral tangent plane and coronal femoral tangent plane and may extend through a largest radius of the femoral medial condyle about the epicondylar axis. Second and third sagittal femoral intersect planes may extend through a smallest radius of the trochlear groove and a largest radius of the lateral femoral condyle, respectively. A contact plane is defined by the transverse femoral tangent plane rotated clockwise from a lateral-directed perspective by 10° or more and positioned on a surface contour of the medial femoral condyle intersected by the first sagittal femoral intersect plane. Control points may be distributed on an active flexion range on articular features of the femur. The articular features of the femur may be any one of or any combination of a medial femoral condyle, a trochlear groove, and a lateral femoral condyle. For the medial femoral condyle, for example, one of the control points may be located on the surface of the femur where the contact plane passes through the surface.

Similar principles to those described above may also be applied to establish reference frames for the tibia. In particular, the same axes and planes as described above may be defined relative to a proximal end of the tibia to define a tibial mechanical reference frame. The articular features of the tibia may include one or both of a medial tibial condyle and a lateral tibial condyle. Three control points may be placed on each articular feature. In sum, the various articular features in the joint may be evaluated under a hierarchy with some features given more weight than others. In particular, the articular features may be weighted in descending order as follows: medial femoral condyle, trochlear groove, medial tibial condyle, lateral tibial condyle and lateral femoral condyle.

Turning to specific details for the identification of control points, the femoral control points may be placed within the active flexion range by reference to the tangent planes and intersect planes or other features within the knee. For example, a first control point may be placed on a surface of each of the medial femoral condyle and lateral femoral condyle at a location tangent to or nearest to the contact plane (e.g., plane at 10 degree angle with respect to transverse femoral tangent plane). Third control points may be placed on the surface of each of the medial femoral condyle and lateral femoral condyle at a location tangent to or nearest to the coronal femoral tangent plane. Second control points may be placed on the surface of each of the medial femoral condyle and the lateral femoral condyle at a location approximately halfway between the respective first and third control points. Each of these points is located on an active flexion portion of the condyle surface. In some arrangements, a series of three control points may be placed on the surface of each of the medial femoral condyle and lateral femoral condyle at locations near to or intersected by the respective one of the sagittal femoral intersect planes and on a plane between 10° and 110° clockwise away from the transverse femoral tangent plane when viewed from a lateral directed perspective, an angular range that corresponds to the active flexion range 33. First, second, and third further control points may be placed on the surface of the trochlear groove at locations intersected by the second sagittal plane and representing approximately 30°, 50°, and 70° of knee flexion away from full extension, respectively. Alternatively, the first, second, and third control points may be placed on the trochlear groove at locations intersected by the second sagittal intersect plane and between 0° and 90° counterclockwise away from the distal most point of the respective condyle from a lateral directed perspective, an angular range that falls within the active flexion range 33. In some examples, the active flexion range may vary from that described above based on the characteristics of the bone of the patient. First, second, and third control points may also be placed on the surface of each of the medial and lateral tibial condyles approximately one third, one half, and two thirds of the way, respectively, from the anterior most point to the posterior most point of the respective condyle.

The articular features may be characterized by fitting equations to the control points on each articular feature. The equations may be fit to the control points by use of a regression. The regression may be a least squares regression. The fit equations may be used to create best fit curves for the articular features. Disease states may be accounted for in deriving the best fit curves from the fit equations. An articular implant may be constructed or selected to match the best fit curves. The articular surfaces may be considered in a pre-defined order to prioritize fit of certain features over others. The best fit of the medial femoral condyle may be considered first. The best fit of the trochlear groove may be considered second. The best fits of the medial tibial condyle, lateral tibial condyle, and lateral femoral condyle may be considered third, respectively. In considering the best fit of each articular feature in sequence, the implant, resection location, and/or intervention type best fitting to the feature at hand may be determined subject to the determinations already made with regard to any determinations made with regard to features of higher priority. For example, proper implant size and resection depth to optimally match the best fit curve established for the medial femoral condyle may be determined without regard for fit of the other articular features. Moreover, the optimal implant size and resection depth to match the best fit curve of the trochlear groove may be determined subject to, and without disturbing, the determinations made for the medial femoral condyle. Next, the optimal implant size and resection depth to match the best fit curve of the medial tibial condyle may be determined subject to, and without disturbing, the determinations made for the medial femoral condyle and the trochlear groove, and so on.

Accounting for disease states in the pre-operative joint may enable approximation of the features of the joint in a healthy state. Such an approximation may enable selecting implants and resection locations to imitate the function of the joint before the disease state set in. Further, prioritizing the articular features by their influence on the function of the joint as a whole, and their proximity to various ligaments, may facilitate treatment that keeps the ligaments within acceptable tension ranges.

In another aspect, a method of optimizing a size of an articular implant may include selecting a plurality of medial femoral condyle control points on a medial femoral condyle of the femur, and mathematically fitting a first medial femoral condyle curve to the plurality of medial femoral condyle control points. The method may further include determining whether any of the plurality of medial femoral condyle control points exceed a threshold deviation from the first medial femoral condyle curve.

In some arrangements according to any of the foregoing, mathematically fitting the first medial femoral condyle curve may include mathematically determining which of a plurality of predetermined implant geometries includes a femoral implant medial condyle that best fits the medial femoral condyle control points.

In some arrangements according to any of the foregoing, each medial femoral condyle control point may be on a surface of the medial femoral condyle of the model and in a sagittal plane, each medial femoral condyle control point being identified based on a distinct angle of knee flexion.

In some arrangements according to any of the foregoing, the angle of knee flexion may be measured based on an orientation of the femur relative to a transverse plane normal to a mechanical axis of a tibia paired with the femur, and the angle of knee flexion for the respective medial femoral condyle control points is 10°, 50°, and 90° of knee flexion, respectively.

In some arrangements according to any of the foregoing, the method may include locating Blumensaat's line on the model, and evaluating a distance between Blumensaat's line and an offset line extending parallel to Blumensaat's line through an inferior point on a trochlear groove of the femoral implant at a planned post-operative implanted position of the femoral implant.

In some arrangements according to any of the foregoing, the method may include selecting the femoral implant and the post-operative implanted position of the femoral implant based on the distance between the offset line and Blumensaat's line and the first medial femoral condyle curve.

In some arrangements according to any of the foregoing, selecting the femoral implant and the post-operative implanted position of the femoral implant may also be based on a comparison of three trochlear groove control points on a trochlear groove of the model to points on the trochlear groove of the implant.

In some arrangements according to any of the foregoing, the three trochlear groove control points may correspond to a surface of the trochlear groove of the femoral model and pass through a single sagittal plane, each of the trochlear groove control points being based on a contact point between the femur and the tibia at a particular angle of knee flexion, the angle of knee flexion being different for each of the trochlear groove control points.

In some arrangements according to any of the foregoing, the three trochlear groove control points may include a first trochlear groove control point established based on a 30 degree angle of knee flexion, a second trochlear groove control point established based on a 50 degree angle of knee flexion, and a third trochlear groove control point established based on a 70 degree angle of knee flexion.

In some arrangements according to any of the foregoing, the method may include, after selecting a femoral implant, selecting a tibial implant and tibial resection depth such that the post-operative range of motion of the knee is on a single sagittal plane, wherein a medial tibial implant condyle contact point of the tibial implant remains in contact with a femoral implant medial condyle of a selected femoral implant throughout the post-operative range of flexion.

In some arrangements according to any of the foregoing, the threshold deviation may be 1.5 mm.

In some arrangements according to any of the foregoing, the method may include identifying any medial femoral condyle control points that exceed the threshold deviation from the first medial condyle curve as irregular.

In some arrangements according to any of the foregoing, the method may include mathematically fitting a second medial femoral condyle curve to the plurality of medial femoral condyle control points with less weight is given to the irregular medial femoral condyle control point.

In some arrangements according to any of the foregoing, the method may include selecting a tibial resection angle measured in a coronal plane. A tibial resection depth and angle may be selected from within a tibial resection depth range and a tibial resection angle range, respectively, in view of a location and diameter of a partial-circular portion of the second medial femoral condyle curve between a subchondral surface of the medial femoral condyle and a medial femoral condyle cartilage surface. A lower end of the tibial resection depth range and a lower end of the tibial resection angle range may correspond to alignment of the partial-circular portion of the second medial femoral condyle curve with the subchondral surface of the medial femoral condyle, and wherein an upper end of the tibial resection depth range and the upper end of the tibial resection angle range correspond to alignment of the partial-circular portion of the second medial femoral condyle curve with the medial femoral condyle cartilage surface.

In some arrangements according to any of the foregoing, the method may include, after selecting a tibial implant and tibial resection depth and angle, selecting a femoral implant lateral femoral condyle size to maintain acceptable tension in lateral ligaments connecting the femur to the tibia throughout the post-operative range of flexion. Typical disease or injury states in a knee affect cartilage more than a subchondral surface of the condyles. Because it is usually possible to infer a pre-injury or pre-disease cartilage thickness, it is possible to approximate the pre-injury or pre-disease tension of soft tissues around the knee at various degrees of flexion by locating the tissue attachment points and accurately characterizing pre-operative subchondral contours of the condyles of the involved bones, particularly the distal femoral condyles. Attempting to recreate the pre-injury or pre-disease soft tissue tensions at various degrees of flexion, including full extension and particularly in the active flexion range, is one factor considered in optimizing implant shape, size, and placement.

In some arrangements according to any of the foregoing, the method may include acquiring a three dimensional scan of a portion of a femur, and generating a computer model of the portion of the femur. The computer model may be used for locating features of the portion of the femur, including for placement of the control points on the femur.

In another aspect, a method of optimizing a size and an implanted position of a femoral implant for use in a knee joint of a patient may include automatically selecting a first set of three points on a medial condyle of the femur within a first plane normal to an axis defined relative to features of the femur, and a second set of three points on a trochlear groove of the femur within a second plane normal to the axis, and using the first and second sets of points, approximating a radius of the medial condyle and a radius of the trochlear groove, respectively. The method may further include determining, using the approximation of the radii of the medial condyle and the trochlear groove, a size of a femoral implant to be implanted onto a prepared distal end of the femur and a position of the femoral implant with respect to the axis.

In some arrangements according to any of the foregoing, the step of automatically selecting may further include determining a desired alignment of the knee joint, and, using data representative of the desired alignment, automatically selecting the first and second sets of points.

In some arrangements according to any of the foregoing, determining the desired alignment may include utilizing a statistical model.

In some arrangements according to any of the foregoing, the desired alignment may be a pre-injury alignment.

In some arrangements according to any of the foregoing, determining the position of the femoral implant with respect to the epicondylar axis may include least squares optimization of a surface of the femoral implant relative to the first and second sets of points.

In some arrangements according to any of the foregoing, the method may include determining a size and position of a tibial implant based in part on a comparison between a first location defined by a surface of a femoral implant medial condyle resulting from the determined sized and location of the femoral implant and a second location defined by a pre-operative subchondral surface of the medial condyle of the femur.

In some arrangements according to any of the foregoing, the method may include determining an optimum size of a femoral implant lateral condyle out of a predefined group of available femoral implants based on the determined size and position of the tibial implant.

In some arrangements according to any of the foregoing, the method may include acquiring a three dimensional scan of a portion of a femur, and generating a computer model of the portion of the femur. The computer model may be used for locating features of the portion of the femur, including for placement of the first and second groups of points on the femur.

### BRIEF DESCRIPTION OF THE DRAWINGS

FIG. 1 is a close up view of an anterior portion of a distal femur.

FIG. 2A is a side view showing a portion of the femur of FIG. 1 viewed in a lateral direction.

FIG. 2B represents approximating geometry overlaid on the view of FIG. 2A.

FIG. 2C is a portion of the femur of FIG. 1 viewed in a superior direction.

FIGS. 3A and 3B are views of the portion of the femur of FIG. 1 from a posterior and anterior direction, respectively, with control points distributed thereon.

FIGS. 3C and 3D are views of the portion of the femur of FIG. 1 in a lateral direction with reference planes superimposed thereon.

FIG. 4A is a view of a proximal portion of a tibia.

FIG. 4B is a view of the portion of the tibia of FIG. 4A in a posterior direction.

FIG. 5A is a side elevation view of the femur of FIGS. 1-3D and the tibia of FIGS. 4A and 4B in a resected state.

FIG. 5B is a flowchart of steps in a method for planning the resections shown in FIG. 5A.

FIGS. 6A-6C are side elevation views of the femur and tibia of FIG. 5A in a post-operative state at greater to lesser degrees of flexion.

### DETAILED DESCRIPTION

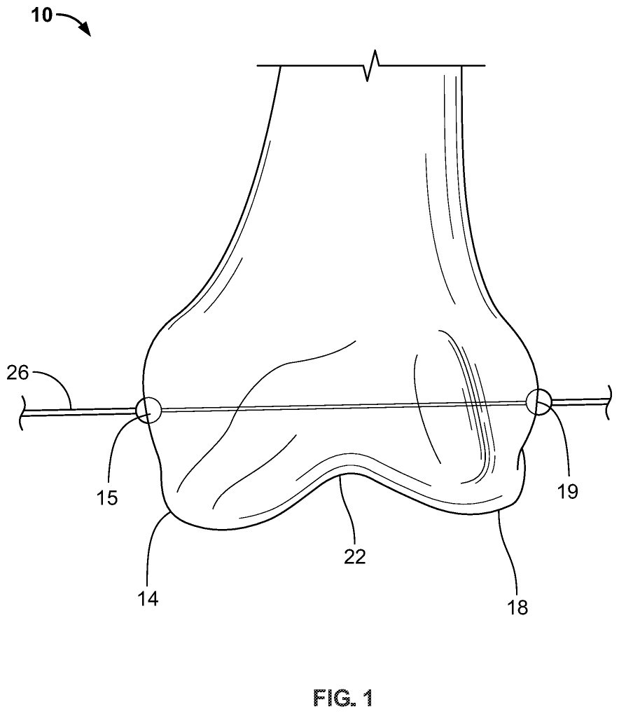

The present disclosure generally relates to pre-operative planning techniques to optimize implant size and placement in a joint. In one aspect, a pre-operative planning technique is employed for knee surgery. A particular embodiment of the technique is shown in FIGS. 1-6C and described as follows. A distal end of an exemplary femur 10 is illustrated in FIG. 1. The distal end of the femur 10 has articular features, including a medial femoral condyle 14, a lateral femoral condyle 18, and a trochlear groove 22 defined between the condyles 14, 18. A femoral epicondylar axis 26 extends through a medial femoral epicondyle 15 and lateral femoral epicondyle 19, located proximally of the medial femoral condyle 14 and lateral femoral condyle 18, respectively.

Portions of the articular features of the femoral condyles 14, 18 of a typical femur 10 can be approximated as portions of relatively simple geometric shapes. Approximating or characterizing a distal end of a femur 10 for a total knee arthroplasty or other surgical intervention can be facilitated by scanning the knee to generate a computer model of the distal portion of the femur 10 approximately as shown in FIG. 1. For knee arthroplasty, the scan covers a tibia and ankle corresponding to the femur 10. The scan data or computer model is used to locate anatomical points, including, for example, center points for the distal femoral head, proximal tibial extremity, knee, and ankle. And, the scan data is used to identify an initial mechanical reference frame that forms the basis for further analysis of the joint. With the femoral head center, the knee center, the ankle center and the proximal tibia center, the initial mechanical reference frames in the form of sagittal, coronal and transverse planes is identified for both the femur and the tibia, thereby establishing a Cartesian coordinate system. Further, as described below, the points derived from the scan data may also be used to establish mechanical axes for the femur and tibia.

Further to the above, the epicondylar and mechanical axes at the distal end of the femur and the proximal end of the tibia may also be used to define femoral and tibial initial mechanical reference frames. This begins with the initial mechanical reference frame as already described, which may be further modified through reference to additional patient anatomical references, such as the epicondylar axis, mechanical axis and posterior and distal condyles of the femur. For each of the femur and the tibia, a transverse plane is defined normal to the respective mechanical axis and along the respective epicondylar axis, a sagittal plane is defined normal to the respective epicondylar axis and parallel to the respective mechanical axis, and a coronal plane is defined along the respective mechanical and epicondylar axes. All other transverse, sagittal, and coronal planes referred to throughout the description below are parallel to the corresponding plane within the relevant femoral or tibial mechanical reference frame.

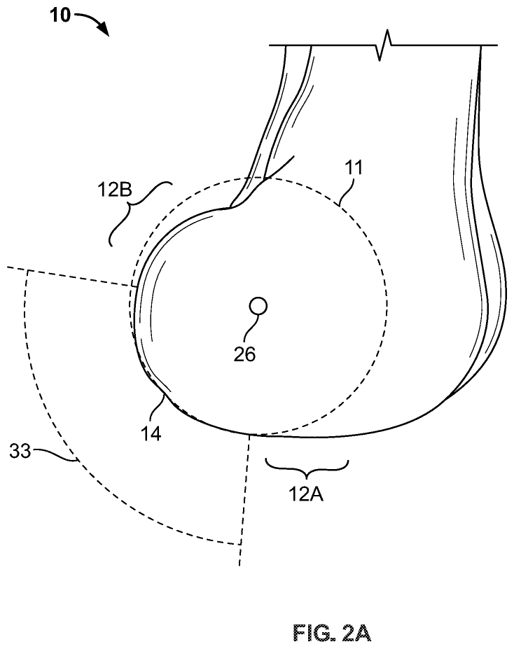

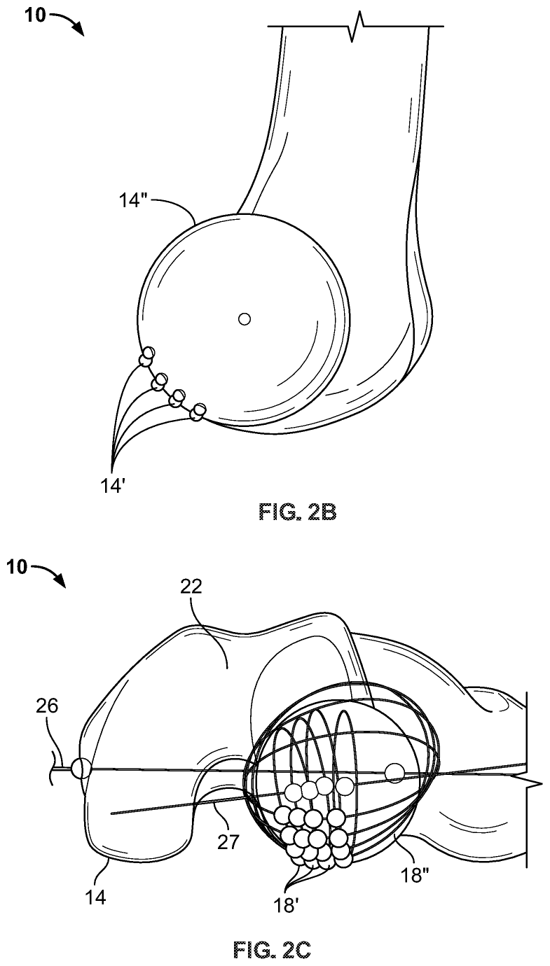

As shown in FIG. 2A, a portion of the medial femoral condyle 14 can be approximated by a circle, illustrated by a reference circle 11 approximately centered on the epicondylar axis 26. The reference circle 11 closely matches a portion of an outer contour of the medial femoral condyle 14 that corresponds generally to a functional range across which a tibia contacts the femur 10 in common degrees of partial flexion of the knee. This range is an active flexion range 33. As shown, the radius of the medial femoral condyle 14 relative to the epicondylar axis 26 increases beyond the active flexion range 33 in an extreme extension range 12A, which prevents the knee from overextending. Similarly, the radius of the medial femoral condyle 14 measured from the epicondylar axis 26 decreases beyond the active flexion range 33 in an extreme flexion range 12B. Thus, the portion of the outer surface of medial femoral condyle 14 between the extreme extension range 12A and the extreme flexion range 12C is the active flexion range.

Because the active flexion range includes points contacted by the tibia during common, day to day usage of the knee, accurate characterization of the active flexion range can contribute significantly to the success of a knee arthroplasty and the post-operative comfort of the patient. Successful characterization of the active flexion range may be accomplished by fitting a curve to a small number of points along the bone surface because of the range's generally partial-circular profile, and choosing a prosthesis and resection location to recreate the pre-operative or pre-injury function of the curve. However, such points must be selected appropriately. Consideration of a point in or near the extreme extension range 12A may result in an approximating curve with a greater radius than the reference circle 11, and consideration of a point in or near the extreme flexion range 12B may result in an approximating curve with a smaller radius than the reference circle 11. Thus, although the extreme extension range 12A and extreme flexion range 12B are points on the medial femoral condyle that may be contacted by the tibia during normal usage, it is advantageous to de-emphasize or avoid these ranges in defining a curve representing the parts of the medial femoral condyle most frequently engaged during typical physical activity. Similar considerations apply to other articular features within a knee, including the lateral femoral condyle 18, trochlear groove 22, and tibial condyles. Each articular feature includes regions that are most commonly engaged during motion of the knee and may be characterized with simple geometric shapes if care is taken to exclude points only engaged in extremes of extension or flexion.

A first array of points 14′ measured on an articular surface of the medial femoral condyle 14 lie approximately along a portion of a first sphere 14″, as shown in FIG. 2B. Similarly, a second array of points 18′ measured on an articular surface of the lateral femoral condyle 18 lie approximately along a portion of a second sphere 18″ as shown in FIG. 2C. A section of the second sphere 18″ to the articular surface of the lateral femoral condyle 18 is lateral to a mid-line of the lateral femoral condyle 18. A portion or section of the surface of the second sphere 18″ fits with a lateral offset within the medial and lateral width of the lateral femoral condyle 18″. The medial radii of that section of the second sphere 18″ are smallest, increasing toward the lateral ⅓ of the lateral femoral condyle 18″, before reducing slightly at the lateral edge of the lateral femoral condyle 18″. In an alternative, the articular surface of the lateral femoral condyle 18 can be approximated as a portion of a cone centered on a skew axis 27 that extends at an angle relative to the epicondylar axis 26. The trochlear groove 22 approximately defines a circular arc.

With each of the articular surfaces approximating a simple geometric shape, and with the relative sizes and locations of a femur 10 typically adhering to predictable patterns, it is possible to characterize the articular features of the distal portion of the femur 10 based on a small number of reference points measured on the surface of the femur 10. Specifically, the size and location of a sphere, cone, or cylinder on which any articular surface may be assumed to lie can be determined from three points.

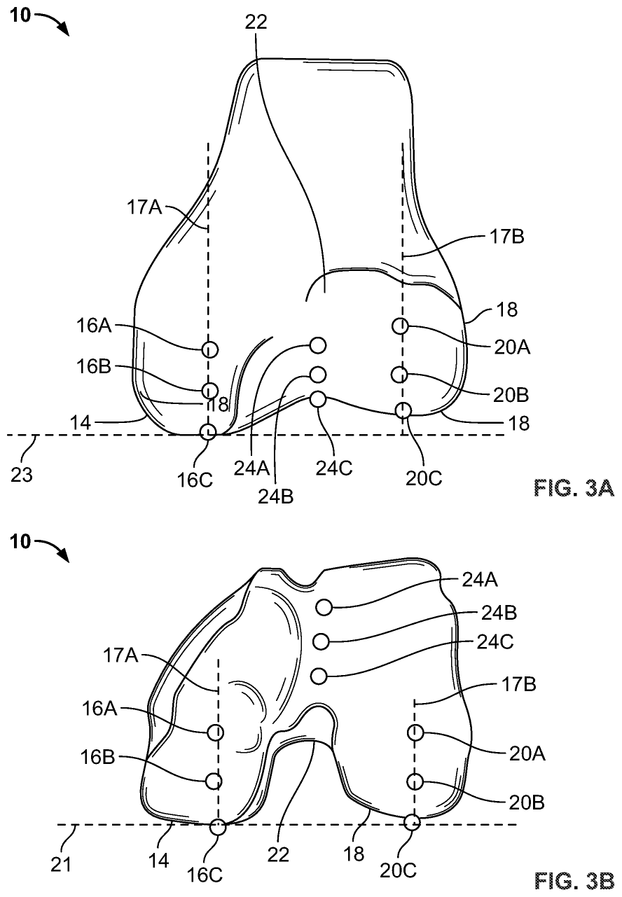

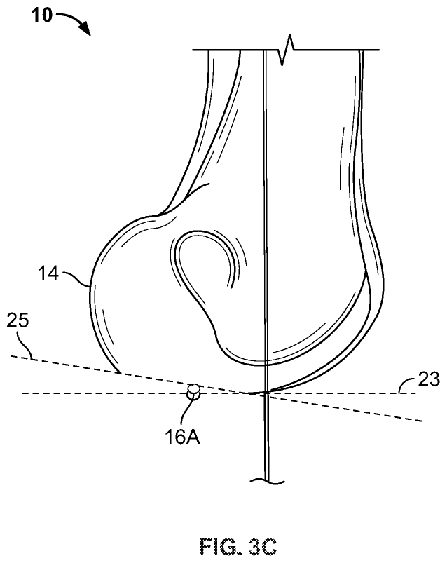

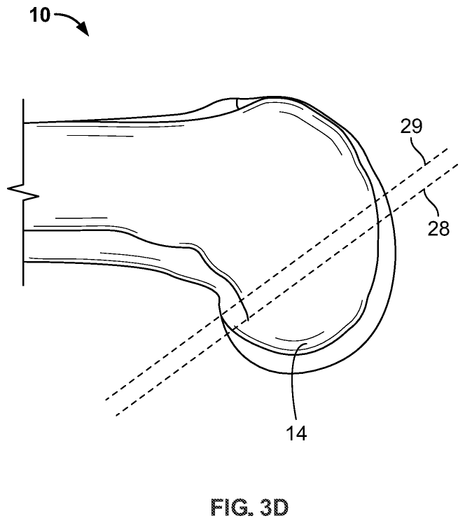

For example, FIGS. 3A and 3B show the distal portion of the femur 10 with three control points on each of the medial femoral condyle 14, lateral femoral condyle 18, and trochlear groove 22. Specifically, first, second, and third medial femoral condyle control points 16A, 16B, 16C, first, second, and third lateral femoral condyle control points 20A, 20B, and 20C, and first, second, and third trochlear groove control points 24A, 24B, and 24C are distributed on the articular surfaces of the corresponding articular features of the femur 10. The medial femoral condyle control points 16A, 16B, 16C and the lateral femoral condyle control points 20A, 20B, 20C are visible through the femur 10 in FIG. 3A, meaning that they are located on the posterior surfaces of their respective condyles and rendered through the bone to be visible from the anterior perspective of FIG. 3A.

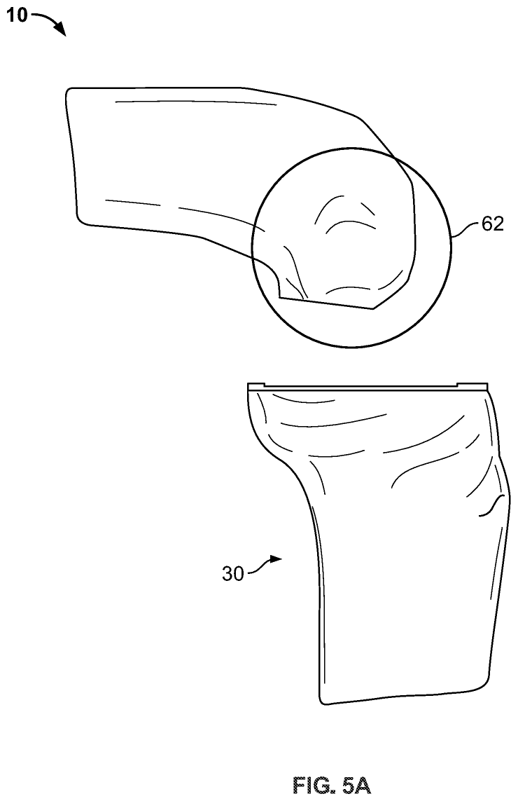

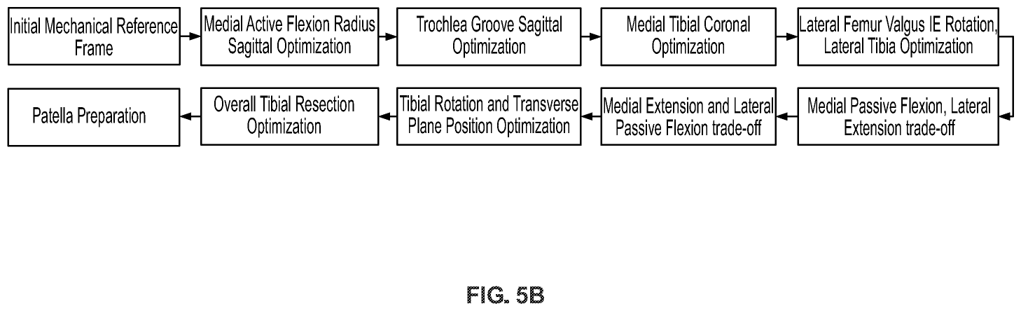

The control points shown in FIGS. 3A and 3B may be located, for example, by use of computer aided scanning or tomography of a patient's knee joint to model the femur 10, then placing the control points on the model. The control points may be placed in order of priority of the underlying articular features to establish the choice of implant. For example, in some arrangements, the control points are placed on the medial femoral condyle 14 first, then the trochlear groove 22, followed by the medial tibial condyle 34, then the lateral tibial condyle 38, and finally the lateral femoral condyle 18. The order of this process is shown in FIG. 5B and described in greater detail below. In some arrangements the locations of the control points may correspond to contact points between the femur 10 and a tibia 30 at various degrees of flexion. In some arrangements, reference planes may be established relative to the model of the femur 10 and tibia 30, and the control points are placed on the surface of the femur 10 and tibia 30 at locations determined relative to where tangent planes and intersect planes are tangent to the respective bone. These planes, e.g., planes of the femoral mechanical reference frame and the tibial mechanical reference frame, are described in the following paragraphs.

A femoral coronal tangent plane 21, visible into the page in FIG. 3B, a view looking in the superior direction, is defined by translating the coronal plane of the femoral initial mechanical reference frame to be tangent to a posterior-most point of either the medial femoral condyle 14 or medial tibial condyle 34. A femoral distal tangent plane 23, visible into the page in FIG. 3A in a view looking in the posterior direction, is defined by translating the transverse plane of the femoral initial mechanical reference plane to be tangent to the distal most point on the medial femoral condyle 14. The femoral coronal tangent plane 21 and the femoral distal tangent plane 23 are thus parallel to the coronal and transverse plane, respectively, as defined relative to the epicondylar axis as discussed above.

A contact plane 25 is defined by the femoral distal tangent plane 23 being rotated 10°, or clockwise from the perspective of FIG. 3C, within a sagittal plane along the surface of the medial femoral condyle 14. The aforementioned planes including the femoral coronal tangent plane 21 and the contact plane 25 constitute the femoral mechanical reference frame.

Returning to the evaluation of the anatomy, we begin with the medial condyle. A cylinder is calculated to fit to the femoral medial condyle control points 16A, 16B, 16C by mathematically defining a best fit circle to the femoral medial condyle control points 16A, 16B, 16C. In some arrangements, the best fit circle is defined with a least squares regression.

In an exemplary arrangement, the first femoral medial condyle control point 16A is placed on a point on the surface of the medial femoral condyle 14 tangent to the contact plane 25 as shown in FIG. 3C, which is representative of approximately 10 degrees of flexion in the joint. The third femoral medial condyle control point 16C is placed on the point of the medial femoral condyle 14 tangent to or nearest to the femoral coronal tangent plane 21 as shown in FIG. 3B, which is representative of approximately 90 degrees of flexion in the joint. The first medial femoral condyle control point 16A and third femoral medial condyle control point 16C thereby approximate contact points between the femoral medial condyle 14 and the tibial medial condyle 34 at approximately 10° and 90° of knee flexion, respectively. The second femoral medial condyle control point 16B is placed approximately halfway between the first femoral medial condyle control point 16A and third femoral medial condyle control point 16C along the surface of the femoral medial condyle 14, thus approximating a contact point between the femoral medial condyle 10 and the tibial medial condyle 34 at 50° flexion of the knee. It should be appreciated that each of these points is located within active flexion range 33, on the active flexion surface.

The femoral medial condyle control points 16A, 16B, 16C typically fall on or near a partial circle on a medial sagittal intersect plane 17A intersecting the medial femoral condyle 14 where its radius relative to the femoral epicondylar axis 26 is greatest, which in various arrangements is modeled as normal to either the mechanical axis of the medial femoral condyle 14 or the epicondylar axis 26. The articular surface of the medial femoral condyle 14 along which the femoral medial condyle control points 16A, 16B, 16C fall can therefore be approximated by a cylinder fit to the partial circle. In optimizing the size and placement of the post-operative articular surfaces of the femur 10 and tibia 30, absolute or squared values of the deviations of the best-fit equation from the control points (e.g., deviation between best-fit curved line and control points) can be used to infer a disease state of the bones or the joint as a whole. For example, in some arrangements, a deviation of 1.5 mm or greater between a control point and a best fit function for any of the condyles 14, 18, 34, 38 or the trochlear groove can be interpreted as an indication of pathology in the bone. In various exemplary arrangements, a sum of the squared deviations of one or multiple of the regressions is calculated to provide a metric for overall pathology of a bone or joint. In further arrangements, one or more individual deviations are considered in isolation, and a new regression is executed with the control points weighted differently to account for acute injuries reflected by the individual deviations. In some examples of such arrangements, a new regression is executed if any individual control point is associated with a deviation is 1.5 mm or more, with that control point weighted less in the new regression. In yet further arrangements, the contour settled upon after one or more regressions is further adjusted to correct for pathologies of the bone and/or the loss of soft tissue as a result of the surgical intervention. For example, if the control points are chosen such that the regression finds a contour along an outer cartilage surface, the equation may be adjusted to follow a contour 2 mm below the outer cartilage surface. In another example, if the pathology of the knee includes a lateral-medial imbalance or misalignment, equations corresponding to one or more of the articular features may be adjusted to restore a pre-disease alignment to the knee.

After the best fit circle on the medial sagittal intersect plane 17A for the femoral medial condyle 14 is derived, a line extending through the center of the best fit circle of the medial femoral condyle 14 is used as a reference to place control points on the trochlear groove 22. In particular, based on rotation of the knee joint about an axis through the center of the best fit circle of the medial femoral condyle, the rotation being within a sagittal plane, points on the trochlear groove are obtained by rotating the joint to various degrees of flexion. These points, the first, second, and third trochlear groove control points 24A, 24B, 24C, shown in FIGS. 3A and 3B, are placed on the trochlear groove 22 at angles representing approximately 30°, 50°, and 70° of flexion, respectively. A location of the control points at these angles may be derived using flexion of the medial condyle as a proxy for flexion in the trochlear groove since flexion is the same for all parts of the distal end of the femur at any point in time. In some examples, the control points on the trochlear groove may be distributed at any locations along the active flexion radius that is characterized by having a generally uniform radius of curvature. A least squares analysis is used to compare the first, second, and third trochlear groove control points 24A, 24B, 24C with three corresponding points on the prosthetic trochlear grooves of available femoral implants. The determination of a best-fit circle for any articular feature may follow the analysis outlined above for the medial femoral condyle 14, including down-weighting of particular control points that appear to deviate significantly from the curve.

Another factor associated with the trochlear groove 22 that is considered for the implant analysis is Blumensaat's line. Blumensaat's line 28 and a trochlear offset line 29, extending parallel to Blumensaat's line 28 and through an inferior point of the trochlear groove 22 are located, as shown in FIG. 3D. The inferior point coincides with the third trochlear groove control point 24C. A distance between Blumensaat's line 28 and the trochlear offset line 29 is measured pre-operatively. Lesser total deviation between such lines is generally preferable, but a minimum distance may be necessary, on a patient-by-patient basis, to maintain adequate soft tissue tension and, by extension, stability of the knee overall. A smaller post-operative distance between Blumensaat's line 28 and the offset line 29 of the femoral implant provides a greater sagittal range of motion for the knee. Conversely, a larger distance between the lines provides a reduced sagittal range of motion. A deviation between line 28 and 29 is a function of the implant size. The smaller the implant, the lesser the deviation. As part of the prioritized weighting of different parts of the knee to properly size and place an implant, the analysis of the trochlear groove, including total deviation between the prosthetic and pre-operative trochlear groove 22 and the minimization of the distance between Blumensaat's line 28 and the post-operative offset line 29, are given less weight in implant selection and resection placement than the analysis of the femoral medial condyle 14.

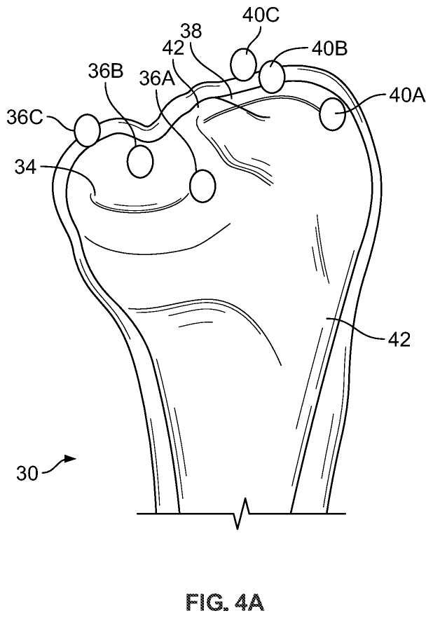

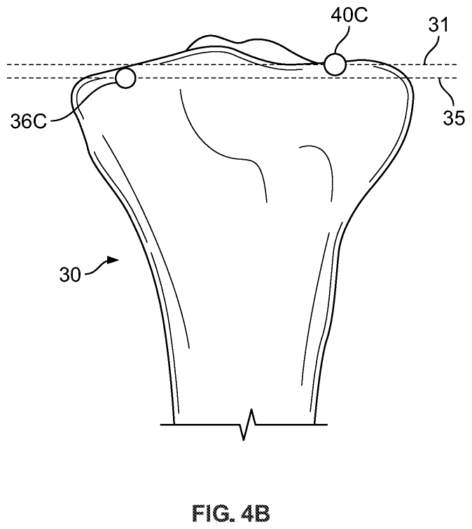

Turning to FIG. 4A, a model is also generated of a proximal end of a tibia 30 that complements the distal end of femur 10 shown in FIG. 1. The tibia 30 includes a medial tibial condyle 34, a lateral tibial condyle 38, and a tibial tubercle 42. A tibial transverse intersect plane 31, shown in FIG. 4B, is defined normal to the mechanical axis of the tibia 30 and intersecting a third lateral tibial condyle control point 40C, which is defined at a point on the lateral tibial condyle 38 that is two thirds of the way from an anterior most point of the lateral tibial condyle 38 to a posterior most point of the lateral tibial condyle 38.

A tibial offset plane 35 is parallel to tibial transverse intersect plane 31 and is defined normal to the mechanical axis of the tibia 30 and intersecting a third medial tibial condyle control point 36C, which is defined at a point on the medial tibial condyle 34 that is two thirds of the way from an anterior most point of the medial tibial condyle 34 to a posterior most point of the medial tibial condyle 34. A distance between the tibial transverse intersect plane 31 and the tibial offset plane 35 can be used to determine an appropriate varus/valgus tilt of the tibial resection. Additionally, the target size and location of the femoral medial condyle 14 as derived from fitting a circle to the femoral medial condyle control points 16A, 16B, 16C may also be used to determine the tilt. The distance between the tibial transverse intersect plane 31 and the offset plane 35 is used in view of the width of the tibial condyles 34, 38, to determine a pre-operative tilt of the proximal end of the tibia 30, and the angle of the tibial resection is adjusted to reflect any difference between the pre-operative femoral medial condyle 14 and the best fitting femoral implant.

As shown in FIG. 4A, the first and second medial tibial condyle control points 36A, 36B are defined anterior to the third medial tibial condyle control point 36C, and the first and second lateral tibial condyle control points 40A, 40B are defined anterior to the third lateral tibial condyle control point 40C. The first tibial condyle control points 36A, 40A are defined one third of the way from the anterior most point to the posterior point of their respective condyles 34, 38, and the second tibial condyle control points 36B, 40B are defined halfway between the corresponding first control points 36A, 40A and third control points 36C, 40C. The placement of the three control points within each tibial condyle relative to each other are considered in determining the target size and location for the tibial implant, including the depth and slope of the tibial resection. Target post-operative locations for points on the tibial implant corresponding to the control points identified in the pre-operative tibial condyles 34, 38 are derived from the pre-operative control points after adjustment for disease states and the probable post-operative size and location of the medial femoral condyle of the femoral implant in view of the considerations described above with regard to optimization of the femoral medial condyle 14 and trochlear groove 22 and with the aim to preserve acceptable tension in the ligaments around the knee. In a manner similar to that described above with regard to the medial femoral condyle 14, best fits for a surface of the medial tibial condyle 34, and then the lateral tibial condyle 38, may be derived from least squares regressions fit to the medial tibial condyle control points 36A, 36B, 36C, and the lateral tibial condyle control points 40A, 40B, 40C, respectively. Successive least squares regressions may be used where an initial regression analysis reveals large individual deviations that may, in some instances, indicate a disease state. In particular, if the control points present deviations over a threshold, such as 1.5 mm, then such control points may be given less weight in subsequent regressions. After final regressions are completed, the size and location of the resulting best fit curves are used to select an appropriate tibial implant and to determine a suitable depth and angle for the tibial resection in view of the dimensions of the tibial implant. In selecting an implant and resection lines for the bone that receives the implant, optimization based on a size and position of the tibial condyles 34, 38 is given less weight than the above described factors concerning the medial femoral condyle 14 and the trochlear groove 22.

Additionally, a calculated resection plane on the tibia is also evaluated to determine whether further consideration of the best-fit of the medial femoral condyle is required. That is, a change in the coronal orientation of the tibial plane post-surgery relative to the orientation prior to surgery is assessed. If it the change is 3 degrees or greater, then a position of the medial femoral condyle is considered to adjust the planned tibial plane. In some arrangements, the best fit circle for the medial femoral condyle control points is used to adjust the angle and depth of the tibial resection from the angle and depth for the tibial resection that would most closely reflect pre-operative anatomy determined in view of the tibial condyle control points. A range of valgus angle adjustment and a range of adjustment to the resection depth at a point below the medial tibial condyle 34 may be predefined to correspond to the location and size of the best fit curve for the medial femoral condyle 14 relative to pre-operative features within the joint. The upper ends of the ranges correspond to the best fit curve for the tibial femoral condyle 14 being aligned with the cartilage covering the pre-operative articular surface of the medial femoral condyle 14, which typically means an offset from the bone of the articular surface of the medial femoral condyle 14 by about 2 mm. The lower ends of the ranges correspond to the best fit curve for the tibial femoral condyle 14 being aligned with the subchondral articular surface of the tibial femoral condyle 14. The smallest angle in the varus angle range and the shallowest medial depth of the medial depth range correspond to the expected post-operative location of the articular surface of the prosthetic medial femoral condyle being aligned with the pre-operative articular surface of the medial femoral condyle 14, and the largest angle in the varus angle range and the deepest medial depth of the medial depth range correspond to the expected post-operative location of the articular surface of the prosthetic medial femoral condyle being aligned with the pre-operative location of the cartilage covering the articular surface of the medial femoral condyle 14, which typically means an offset from the pre-operative articular surface of the medial femoral condyle 14 by about 2 mm. An exemplary varus angle range is from 0° to 3°, and an exemplary resection depth below the medial tibial condyle 34 ranges from 4 mm at a lower end to 2 mm less than a thickness of the selected tibial implant at its medial condyle at an upper end. Thus, the closer the expected post-operative location of the articular surface of the prosthetic medial femoral condyle provided by the femoral implant is to the pre-operative location of the cartilage surface, i.e., the further from the pre-operative location of the subchondral surface of the medial femoral condyle 14, the greater the angle of the tibial resection will be and the deeper the tibial resection will be under the medial tibial condyle 34. This completes the analysis of the tibia.

Next, the analysis returns to the femur with attention to the lateral femoral condyle in particular. As we proceed through the hierarchy of prioritization, the lateral femoral condyle carries less weight than the medial femoral condyle, trochlear groove of the femur and the tibia. First, second, and third lateral femoral condyle control points 20A, 20B, 20C are defined on a lateral sagittal plane 17 extending through the center of the lateral femoral condyle 18 where the sagittal plane intersects the surface of the lateral femoral condyle 18. The first and third lateral femoral condyle control points 20A, 20C are the points at the intersection of the lateral sagittal plane 17 and the surface of the lateral femoral condyle 18 that are closest to the contact plane 25 and the coronal plane 21, respectively. The second lateral femoral condyle control point 20B is a point along the intersection of the lateral sagittal plane 17 that is half way between the first lateral femoral condyle control point 20A and the third lateral femoral condyle control point 20C. A circle is fit to the lateral femoral condyle control points 20A, 20B, 20C using a regression analysis. The regression may be performed in any manner as described elsewhere in the disclosure. Similar to the best fit circle for the medial femoral condyle 14, the best fit circle for the lateral femoral condyle 18 is used to determine a target post-operative location and size for a lateral femoral condyle of a femoral implant. The best-fit analysis of the lateral femoral condyle may also be considered to evaluate the varus-valgus of the knee, in conjunction with the data associated with the other factors. In selecting the implants and resection locations for a procedure, the best fit circle for the lateral femoral condyle is given less weight than the above described factors derived from the medial tibial condyle 34, and therefore less weight than the above described factors derived from the trochlear groove 22 and the medial femoral condyle 14.

In some procedures, the anterior/posterior slope of the tibial resection is determined in view of the desired post-operative range of flexion and tension in soft tissue around the joint. For example, greater downward slope of the tibial resection in the posterior direction enables a greater range of knee flexion, but may increase tension in the patellar tendon. These factors are considered in addition to the location and size of the best fit curves fit onto the control points in the tibial condyles.

In some procedures, anterior medial resection and posterior lateral resection of the tibia is evaluated in detail if it is determined that there is a significant difference between the two based on the plan established through the hierarchical evaluation of the anatomy. Other potential considerations during the procedure may include external-internal rotation of the femur with respect to the tibia. Such consideration may allow for positioning of the implants on the knee to best conform to a natural position. Further, global laxity or medial elasticity may be refined with planar shifts in the tibial resection. The patella may also be taken into consideration through the creation of a resected reference surface(s) that allows the restoration of the thickness of the patella.

After the articular features of one or more bones in a joint characterized, it is possible to plan a surgical intervention and select an appropriate prosthesis to maintain or improve upon pre-operative geometry of the joint. Thus, the approach described above with regard to FIGS. 3A-4B enables planning for treatment of a dysfunctional joint based on relatively few reference points. As an example, FIG. 5A shows a resected femur 10 and tibia 30, with a medial femoral condyle best fit circle 62 superimposed. The diameter and location of the best medial femoral condyle best fit circle 62 result from the above described regression and adjustment process. Though not illustrated, similar best fit curves for the trochlear groove 22, lateral femoral condyle 18, medial tibial condyle 34, and lateral tibial condyle 38 are also derived from the above described regression and adjustment process. Each best fit curve is located relative to features of the femur 10 or tibia 30 that remain after resection, such as the epicondylar axes 26, 46. In cooperation, the best fit circles indicate workable sizes for femoral and tibial implants with little or no need for trialling and gap balancing.

A process for characterizing joint features, selecting implants, and planning resections according to an arrangement is illustrated in FIG. 5B. The medial femoral condyle 14 is typically the most important articular feature in determining the function of a knee joint. For that reason, accurately recreating the medial femoral condyle 14 with the positioning and choice of resections and femoral implant plays a significant role in preserving natural function of the knee. Further, other articular features within a knee joint may be inferred from the size and location of the medial femoral condyle 14 or may be altered without affecting the function of the knee if the characteristics of the medial femoral condyle 14 and other dependent features are not changed, or are replaced with functional equivalents. For those reasons, in some arrangements, a method for planning an intervention and selecting femoral and tibial implants prioritizes characterizing and recreating the function of the medial femoral condyle 14 over other features within the knee. In further arrangements, the trochlear groove 22 is prioritized immediately after the medial femoral condyle 14 because, among other reasons, the proper size for a femoral implant can be refined based on aa pre-operative and post-operative distance between Blumensaat's line 28 and the offset line 29. In further arrangements, the medial tibial condyle 34, lateral femoral condyle 18, and lateral tibial condyle 38, are prioritized, in order, behind the trochlear groove 22.

In some such arrangements, decisions such implant size and placement and resection depth and angle made to fit the curve of each articular feature are made subject to the decisions already made with regard to other features of earlier priority. For example, a femoral resection depth at the medial side may be determined, and one or more suitable implants may be selected, to match the best fit curve generated for the femoral medial condyle 14 before consideration of the best fit curves for the other articular features. The resection angle and suitable implants may be further narrowed down to match the best fit curve of the trochlear groove 22 as well as possible without disturbing the decisions made for matching the best fit curve of the medial femoral condyle 14. Similarly, implant and resection are made to match the best fit curve of the tibial femoral condyle 34 as well as possible from within the available solutions that will cooperate effectively with the post-operative state of the distal femur as dictated by the choices already made for matching the best fit curves of the medial femoral condyle 14 and trochlear groove 22. Further, choices made to match the best fit curve of the lateral tibial condyle 38 are then made without altering the determinations already made with regard to the medial femoral condyle 14, trochlear groove 22, and medial tibial condyle 34, and finally choices are made to match the best fit curve of the lateral femoral condyle 18 without altering the decisions already made with regard to any of the preceding four articular features.

The above described prioritization can be altered if deemed necessary by a surgeon. For example, it may be appropriate to prioritize the five articular features in a different order than that set out above in usual cases, such as where disease, injury, or prior surgery has altered the knee significantly from typical anatomy. In another example, it may be appropriate to adjust determinations with regard to earlier-priority articular features in view of findings with regard to later-prioritized articular features where a knee presents particularly atypical anatomy. In such arrangements, deviations from the best fit at each of the articular features are balanced against one another, with the articular features weighted according to their priority.

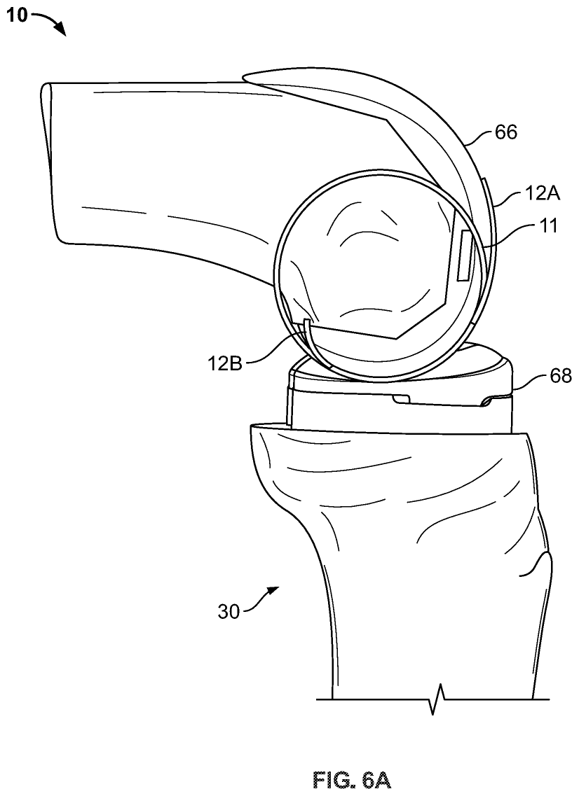

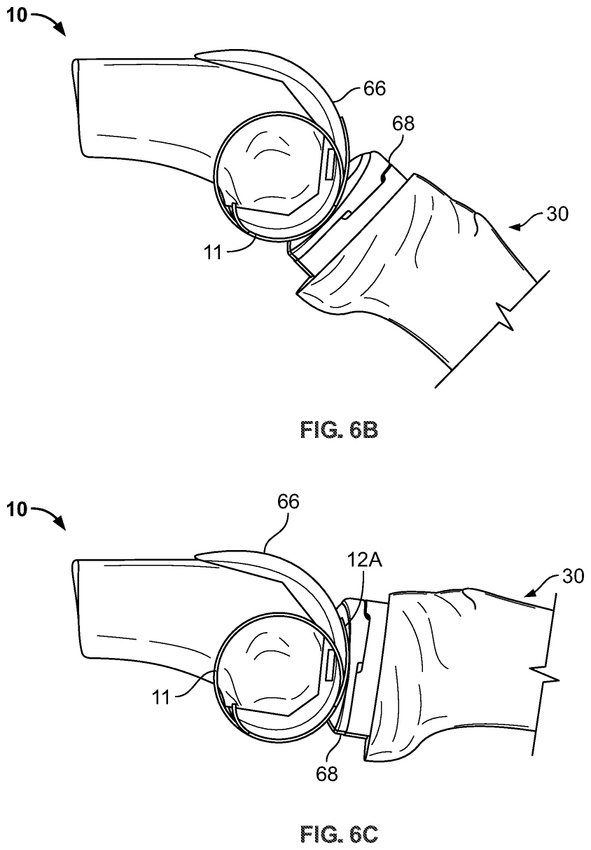

The tibial component can be selected in view of the anticipated post-operative size and location of medial femoral condyle of the femoral implant to have a medial tibial condyle that will cooperate with the femoral implant to achieve a desired range of motion for the knee. As shown in FIG. 6A-6C, a femoral implant 66 and tibial implant 68 may be selected and implanted on resected surfaces of the femur 10 and tibia 30, respectively, according to the prioritized decision making described above. Throughout the active flexion range, encompassing the two positions shown in FIGS. 6A and 6B, the prosthetic medial femoral condyle of the femoral implant 66 approximately follows the reference circle 11 as defined with regard to the pre-operative and, ideally, pre-injury state of the femur 10 as a result of matching the implant size, shape, and location to the best fit curves generated from the control points. A prosthetic medial tibial condyle provided by the tibial implant 68 permits smooth articulation of the prosthetic medial femoral condyle throughout the active flexion range. The articulation between the prostheses 66, 68 throughout the active flexion range will likely apply a comfortable degree of tension on the soft tissue around the knee as a result of the above described planning steps. Because femoral prostheses 66 usually imitate typical knee anatomy, the prosthetic medial femoral condyle will fit the contour of the pre-operative extreme extension range 12A to about the same degree that the prosthetic medial femoral condyle fits the reference circle 11 throughout the active flexion range. The post-operative knee should therefore be similarly prevented from overextension. Larger deviations are tolerable from the contour of the pre-operative extreme flexion range 12B because the extreme flexion range 12B sees relatively little engagement in common physical activities.

In various arrangements, selecting an implant for the femur 10 or tibia 30 includes choosing an implant from a preexisting group of implants that best matches the femur 10 or tibia's 30 best fit curves or constructing a patient-specific implant to match the best fit circles. Selecting a femoral implant 66 and tibial implant 68 this way permits the selection of implants that imitate the articular features of the joint in a pre-operative and disease free state, comfortably retaining much of the joint's original range of motion as illustrated in FIGS. 7A and 7B. In other embodiments, the method may be employed so that the results obtained may be used to manufacture patient specific implants.

Although the concepts herein have been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of optimizing a size of an articular implant based on a patient's anatomy, the method comprising:
selecting a plurality of medial femoral condyle control points on a medial femoral condyle of a femur;
mathematically fitting a first medial femoral condyle curve to the plurality of medial femoral condyle control points;
determining whether any of the plurality of medial femoral condyle control points exceed a threshold deviation from the first medial femoral condyle curve.

2. The method of claim 1, wherein mathematically fitting the first medial femoral condyle curve is mathematically determining which of a plurality of predetermined implant geometries includes a femoral implant medial condyle that best fits the medial femoral condyle control points.

3. The method of claim 1, further comprising identifying any medial femoral condyle control points that exceed the threshold deviation from the first medial condyle curve as irregular.

4. The method of claim 3, wherein none of the plurality of medial femoral condyle control points are identified as irregular.

5. The method of claim 3, wherein when a first medial femoral condyle control point of the plurality of medial femoral condyle control points is identified as irregular, mathematically fitting a second medial femoral condyle curve to the plurality of medial femoral condyle control points with less weight is given to the first medial femoral condyle control point.

6. The method of claim 5, wherein each medial femoral condyle control point is on a surface of the medial femoral condyle of the model and in a sagittal plane, each medial femoral condyle control point being identified based on a distinct angle of knee flexion.

7. The method of claim 6, wherein the angle of knee flexion is measured based on an orientation of the femur relative to a transverse plane normal to a mechanical axis of a tibia paired with the femur, and the angle of knee flexion for the respective medial femoral condyle control points is 10°, 50°, and 90° of knee flexion, respectively.

8. The method of claim 5, further comprising locating Blumensaat's line on the model, and evaluating a distance between Blumensaat's line and an offset line extending parallel to Blumensaat's line through an inferior point on a trochlear groove of the femoral implant at a planned post-operative implanted position of the femoral implant.

9. The method of claim 8, further comprising selecting the femoral implant and the post-operative implanted position of the femoral implant based on the distance between the offset line and Blumensaat's line and the first medial femoral condyle curve.

10. The method of claim 9, wherein selecting the femoral implant and the post-operative implanted position of the femoral implant is also based on a comparison of three trochlear groove control points on a trochlear groove of the model to points on the trochlear groove of the implant.

11. The method of claim 10, wherein the three trochlear groove control points correspond to a surface of the trochlear groove of the femoral model and pass through a single sagittal plane, each of the trochlear groove control points being based on a contact point between the femur and the patella at a particular angle of knee flexion, the angle of knee flexion being different for each of the trochlear groove control points.

12. The method of claim 6, wherein the three trochlear groove control points include a first trochlear groove control point established based on a 30 degree angle of knee flexion, a second trochlear groove control point established based on a 50 degree angle of knee flexion, and a third trochlear groove control point established based on a 70 degree angle of knee flexion.

13. The method of claim 10, further comprising, after selecting a femoral implant, selecting a tibial implant and tibial resection depth such that the post-operative range of motion of the knee is on a single sagittal plane, wherein a medial tibial implant condyle contact point of the tibial implant remains in contact with a femoral implant medial condyle of a selected femoral implant throughout the post-operative range of flexion.

14. The method of claim 5, further comprising:
selecting a tibial resection angle measured in a coronal plane,
wherein a tibial resection depth and angle are selected from within a tibial resection depth range and a tibial resection angle range, respectively, in view of a location and diameter of a partial-circular portion of the second medial femoral condyle curve between a subchondral surface of the medial femoral condyle and a medial femoral condyle cartilage surface, and
wherein a lower end of the tibial resection depth range and a lower end of the tibial resection angle range correspond to alignment of the partial-circular portion of the second medial femoral condyle curve with the subchondral surface of the medial femoral condyle, and wherein an upper end of the tibial resection depth range and the upper end of the tibial resection angle range correspond to alignment of the partial-circular portion of the second medial femoral condyle curve with the medial femoral condyle cartilage surface.

15. The method of claim 1, further comprising:
acquiring a three dimensional scan of a portion of a femur; and
generating a computer model of the portion of the femur; and
wherein the computer model is used for locating features of the portion of the femur, including for selection of the plurality of medial femoral condyle control points on the femur.

16. A method of optimizing a size and an implanted position of a femoral implant for use in a knee joint of a patient, the method comprising:
using a three-dimensional computer model of a femur, automatically selecting a first set of three points on a medial condyle of the computer model of the femur within a first plane normal to an epicondylar axis of the computer model of the femur, and a second set of three points on a trochlear groove of the computer model of the femur within a second plane normal to the epicondylar axis;
using the first and second sets of points, approximating a radius of the medial condyle and a radius of the trochlear groove, respectively; and
determining, using the approximation of the radii of the medial condyle and the trochlear groove, a size of a femoral implant to be implanted onto a prepared distal end of the femur and a position of the femoral implant with respect to the epicondylar axis.

17. The method of claim 16, further comprising:
wherein the step of automatically selecting includes determining a desired alignment of the knee joint, and using data representative of the desired alignment, automatically selecting the first and second sets of points; and
determining the desired alignment includes utilizing a statistical model.

18. The method of claim 17, wherein the desired alignment is a pre-injury alignment.

19. The method of claim 16, wherein determining the position of the femoral implant with respect to the epicondylar axis includes least squares optimization of a surface of the femoral implant relative to the first and second sets of points.

20. The method of claim 16, comprising determining a size and position of a tibial implant based in part on a comparison between a first location defined by a surface of a femoral implant medial condyle resulting from the determined size and location of the femoral implant and a second location defined by a pre-operative subchondral surface of the medial condyle of the femur.
